(19) United States
(12) Patent Application Publication
(10) Pub. No.: US 2026/0165930 A1
(43) Pub. Date: Jun. 18, 2026

(54) Sunscreen Compositions

(71) Applicant: Colgate-Palmolive Company, New York, NY (US)

(72) Inventors: Devon Sean COCUZZA, Flemington, NJ (US); Erika PAMBIANCHI, Middlesex, NJ (US)

(73) Assignee: Colgate-Palmolive Company, New York, NY (US)

(21) Appl. No.: 19/415,177

(22) Filed: Dec. 10, 2025

Related U.S. Application Data
(60) Provisional application No. 63/733,064, filed on Dec. 12, 2024.

(51) Int. Cl. A61K 8/37; A61K 8/06; A61K 8/27; A61K 8/29; A61K 8/31; A61K 8/35; A61K 8/67; A61Q 17/04

(52) U.S. Cl. CPC A61K 8/37; A61K 8/062; A61K 8/27; A61K 8/29; A61K 8/31; A61K 8/35; A61K 8/678; A61Q 17/04; A61K 2800/30; A61K 2800/52; A61K 2800/522

(57) ABSTRACT

Disclosed herein are water-resistant sunscreen compositions comprising an oil-in-water emulsion, wherein the oil-in-water emulsion comprises at least one UV active ingredient, at least one alkane, and at least one photostabilizer and is substantially free of an emulsifier, and wherein the sunscreen composition provides a water-resistant sun protection factor (SPF) of at least about 30. Also disclosed herein are methods of using these water-resistant sunscreen compositions to prevent or mitigate damage to skin.

### CROSS-REFERENCE TO RELATED APPLICATIONS

This application claims the benefit of priority from U.S. Provisional Patent Application No. 63/733,064, filed 12 Dec. 2024, the contents of which are hereby incorporated herein by reference in their entirety.

### BACKGROUND

The negative effects of exposure to ultraviolet (“UV”) light are well known. Prolonged exposure to sunlight causes damage such as sunburn to the skin and hair. Exposure to UV light at certain wavelengths, from about 290 nm to about 400 nm, for example, may lead to long term damage as well as serious conditions such as skin cancer.

For example, ultraviolet radiation in the shorter wavelength UVB region of 290 to 320 nm is considered significant in causing burning. Accordingly, protection against UVB radiation has traditionally been a primary target of sunscreen formulations. Relatively recently there has also been a desire for further protection against UVA radiation to reduce long-term skin damage. Generally, UV light contributes to aging by causing free radicals, such as O—, OH, superoxide anion, nitric oxide, and hydrogen radicals, to form in the skin. Free radicals attack DNA, membrane lipids, and proteins, generating carbon radicals. These in turn react with oxygen to produce a peroxyl radical that can attack adjacent fatty acids to generate new carbon radicals. The end result is a loss of skin elasticity and the appearance of wrinkles.

More importantly, both the UVA and UVB wavebands have been identified as potential factors for increasing skin cancers, e.g., melanomas. Skin cancer is one of the most common cancer types, and its incidence world-wide is problematic. For these and other reasons, consistent use of a topical sunscreen is now strongly recommended.

Sunscreens are subject to strict regulatory approval by various federal agencies in order to be considered “broad spectrum” and to carry a specified sun protection factor (SPF) rating. For example, to be considered a “broad spectrum” sunscreen, the current federal standard (21 C.F.R. § 201.327) requires that the mean critical wavelength is identified as the wavelength at which the integral of the spectral absorbance curve reaches 90 percent of the integral over the UV spectrum from 290 to 400 nm.

Accordingly, there is a need for sunscreen compositions that can provide adequate protection from UV light and meet the changing regulatory standards, while also providing water-resistance and an acceptable feel.

### BRIEF SUMMARY

In one aspect, the disclosure provides a sunscreen composition, such as a sunscreen composition in the form of an oil-in-water emulsion. In certain embodiments, disclosed herein is a water-resistant sunscreen composition comprising an oil-in-water emulsion, wherein the oil-in-water emulsion comprises at least one UV active ingredient, at least one alkane, and at least one photostabilizer and is substantially free of an emulsifier, and wherein the composition provides a water-resistant sun protection factor (SPF) of at least about 30.

In some embodiments, the at least one UV active ingredient is at least one metal oxide, such as zinc oxide and titanium dioxide, preferably zinc oxide. In certain embodiments the at least one UV active ingredient (e.g., a metal oxide such as zinc oxide) is present in an amount ranging from about 10 wt % to about 20 wt %, preferably from about 13 wt % to about 16 wt %, or from about 15 wt % to about 16 wt %, based on a total weight of the water-resistant sunscreen composition.

In some embodiments of the disclosure, the water-resistant sunscreen composition is free of or substantially free of a film former.

In certain embodiments of the disclosure, the at least one photostabilizer is selected from ethylhexyl methoxycrylene, butyloctyl salicylate, or a combination thereof, and in certain embodiments, the at least one photostabilizer is a combination of ethylhexyl methoxycrylene and butyloctyl salicylate. In certain embodiments, the at least one photostabilizer is present in the water-resistant sunscreen composition in an amount ranging from about 1 wt % to about 15 wt %, such as from about 5 wt % to about 10 wt %, preferably about 7.5 wt % or about 8 wt %, based on a total weight of the water-resistant sunscreen composition. In certain embodiments, ethylhexyl methoxycrylene is present in the sunscreen composition an amount greater than about 2.5 wt %, such as about 3 wt %, based on a total weight of the water-resistant sunscreen.

In various embodiments, the at least one alkane is selected from C15-C19 alkanes, and in various embodiments, the water-resistant sunscreen composition is free of or substantially free of isododecane.

In certain embodiments, the water-resistant sunscreen composition further comprises at least one skin conditioning agent, and in certain embodiments, the at least one skin conditioning agent is selected from amino acids (e.g., glycine, arginine, and/or taurine), polysilicones, bisabolol, and combinations thereof. In certain embodiments, the water-resistant sunscreen composition further comprises at least one antioxidant, and in certain embodiments, the at least one antioxidant is selected from vitamin E and derivatives thereof. In certain exemplary embodiments of the disclosure, the water-resistant sunscreen composition further comprises a cosmetically acceptable carrier, preferably water, and in certain embodiments, water is present in the water-resistant sunscreen composition in an amount ranging from about 40 wt % to about 60 wt %, preferably about 50 wt % to about 55 wt %, based on a total weight of the water-resistant sunscreen composition.

Also disclosed herein are methods of preventing or mitigating damage to skin, the methods comprising applying the water-resistant sunscreen composition disclosed herein to the skin of a user in need thereof and allowing the water-resistant sunscreen composition to dry on the skin before exposing the skin to UV radiation. In certain embodiments of the methods disclosed herein, the methods provide SPF protection to the user in an amount of at least about 30, such as at least about 35, at least about 40, or at least about 45, and in certain embodiments, the skin is exposed to water for a period of time, such as at least about 40 minutes.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspects is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material. As used herein, “substantially free” of a material refers to a composition where the material is present in an amount of less than about 0.1 weight %, such as less than about 0.05 weight %, less than about 0.01 weight %, less than about 0.005 weight %, less than about 0.001 weight %, or less than about 0.0001 weight % based on a total weight of the composition. In certain embodiments, “substantially free” of a material indicates that the material is not present in any detectable amount in the composition.

As used herein, the term “Sun Protection Factor” or “SPF” indicates a measure of the amount of solar energy (UV radiation) that is required to produce sunburn on protected skin, e.g., skin to which sunscreen has been applied. The higher the SPF value, the greater the sunburn protection on the skin.

As used herein, the term “water resistant,” “water resistance,” and the like indicate a composition that provides a stated SPF after immersion in water following the methodology set forth in 21 C.F.R. § 352.76 (2024, U.S. Food and Drug Administration, Department of Health and Human Services). Namely, 21 C.F.R. § 352.76 states that for sunscreen products in the U.S. to make the claim of “water resistant,” the SPF value shall be determined after application of the sunscreen and 40 minutes of water immersion, including 20 minutes moderate activity in water, followed by 20 minutes of rest (without towel drying), and then 20 additional minutes of moderate activity in water.

Disclose herein is a water-resistant sunscreen composition that is an oil-in-water emulsion. In certain embodiments, the sunscreen composition disclosed herein may be free of or substantially free of an emulsifier, and in certain embodiments, the sunscreen composition disclosed herein may be free of or substantially free of a film former. In certain embodiments, the sunscreen composition disclosed herein may be free of or substantially free of both an emulsifier and a film forming polymer. In certain embodiments, the sunscreen composition comprises at least one metal oxide (e.g., zinc oxide), at least one photostabilizer (e.g., ethylhexyl methoxycrylene), at least one alkane (e.g., a C15-C19 linear alkane), and any combination thereof.

In certain embodiments, the water-resistant sunscreen composition disclosed herein may have an SPF of at least about 20, such as at least about 25, at least about 30, at least about 35, at least about 40, at least about 45, or at least about 50. In certain embodiments, the water-resistant sunscreen composition disclosed herein has an SPF ranging from about 15 to about 65, such as about 20 to about 60, about 25 to about 55, about 30 to about 50, about 35 to about 45, about 37 to about 43, about 38 to about 42, about 35, about 36, about 37, about 38, about 39, about 40, about 41, about 42, about 43, about 44, or about 45. The SPF of the sunscreen composition may be determined by any method known in the art. In certain embodiments, the SPF is determined according to the methodology set forth in 21 C.F.R. §§ 352.70-352.73 (2024).

According to certain embodiments disclosed herein, the water-resistant sunscreen composition may be free of or substantially free of an emulsifier, and in certain embodiments, the water-resistant sunscreen composition may comprise at least one emulsifier. The emulsifier, when present, may be chosen from any emulsifier or emulsifying agent known in the art. Exemplary emulsifiers may be selected from amphoteric, anionic, cationic or nonionic emulsifiers, alone or in a mixture, and optionally a co-emulsifier. Emulsifiers may be chosen in an appropriate manner according to the desired emulsion, e.g., oil-in-water emulsion or water-in-oil emulsion.

For water-in-oil emulsions, exemplary emulsifiers may include dimethicone copolyols, such as the mixture of cyclomethicone and dimethicone copolyol, and alkyl dimethicone copolyols such as lauryl dimethicone copolyol and cetyl dimethicone copolyol, as well as crosslinked elastomeric solid organopolysiloxanes comprising at least one oxyalkylene group, such as those disclosed, for example, in U.S. Pat. Nos. 5,412,004 and 5,811,487, the contents of each of which are incorporated herein by reference.

For oil-in-water emulsions, exemplary emulsifiers include those selected from nonionic emulsifiers, such as oxyalkylenated (e.g., polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

The fatty acid esters of a sugar that can be used as nonionic amphiphilic lipids can include esters or mixtures of esters of a C₈-C₂₂ fatty acid and of sucrose, of maltose, of glucose or of fructose, and esters or mixtures of esters of a C₁₄-C₂₂ fatty acid and of methylglucose.

The C₈-C₂₂ or C₁₄-C₂₂ fatty acids forming the fatty unit of the esters that can be used in the emulsion can comprise a saturated or unsaturated linear alkyl chain having, respectively, from 8 to 22 or from 14 to 22 carbon atoms. The fatty unit of the esters can be chosen from stearates, behenates (e.g., glyceryl behenate), arachidonates, palmitates (e.g., isopropyl palmitate), myristates, laurates, caprates and mixtures thereof.

In some aspects, esters or of mixtures of esters of a fatty acid and of sucrose, of maltose, of glucose or of fructose, that can be included or excluded from any of the sunscreen compositions disclosed herein, and can, for example, be selected from sucrose monostearate, sucrose distearate, sucrose tristearate, and mixtures thereof, having an HLB (Hydrophilic Lipophilic Balance) of 5, 7, 11 and 16; methylglucose, distearate of methylglucose polyglycerol-3, glucose monoesters or maltose monoesters, such as methyl O-hexadecanoyl-6-D-glucoside and O-hexadecanoyl-6-D-maltoside, and combinations thereof.

In some aspects, the fatty alcohol ethers of a sugar that can be used in the sunscreen compositions disclosed herein (or excluded from the sunscreen compositions disclosed herein) as nonionic amphiphilic lipids chosen from ethers or mixtures of ethers of a C₈-C₂₂ fatty alcohol and of glucose, of maltose, of sucrose or of fructose, and ethers or mixtures of ethers of a C₁₄-C₂₂ fatty alcohol and of methylglucose.

In certain aspects, the C₈-C₂₂ or C₁₄-C₂₂ fatty alcohols forming the fatty unit of the ethers that can be included or excluded from the emulsion of the sunscreen compositions disclosed herein comprise a saturated or unsaturated linear alkyl chain having, respectively, from 8 to 22 or from 14 to 22 carbon atoms. For example, the fatty unit of the ethers can be chosen from decyl, cetyl, behenyl, arachidyl, stearyl, palmityl, myristyl, lauryl, capryl and hexadecanoyl units, and mixtures thereof such as cetearyl.

In certain aspects, the fatty alcohol ethers of a sugar can be any selected from alkylpolyglucosides, such as decylglucoside and laurylglucoside, cetostearylglucoside, optionally as a mixture with cetostearyl alcohol, and also arachidylglucoside, for example in the form of the mixture of arachidyl and behenyl alcohols and of arachidylglucoside.

In certain aspects, the sunscreen can include or exclude a nonionic amphiphilic lipid of sucrose monostearate, sucrose distearate, sucrose tristearate and mixtures thereof, the distearate of methylglucose and of polyglycerol-3, and alkylpolyglucosides. In certain aspects, the glycerol fatty esters that can be used as nonionic amphiphilic lipids can be chosen from the esters formed from at least one acid comprising a saturated linear alkyl chain having from 16 to 22 carbon atoms, and from 1 to 10 glycerol units. For example, glycerol fatty esters may be used as an emulsifier. Exemplary esters may be chosen from stearates, behenates, arachidates, palmitates, and mixtures thereof.

In other aspects, an emulsifier can be selected from: oleth-20, oleth-3 phosphate, cetearyl alcohol, cetearyl glucoside, coco-glucoside, polyhydroxystearic acid, lauryl glucosider, myristyl glucoside, polyglyceryl-6-laurate, decaglycerol monostearate, distearate, tristearate and pentastearate (10 glycerol units) (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate) such as the product sold by the company Nikko under the name Nikkol DGMS, and mixtures thereof.

In certain embodiments of the disclosure, the sunscreen composition may contain less than about 2 wt % of an emulsifier, such as about 1.5 wt % or less, about 1 wt % or less, less than about 1 wt %, less than about 0.5 wt %, less than about 0.25 wt %, less than about 0.2 wt %, less than about 0.1 wt %, less than about 0.05 wt %, less than about 0.001 wt %, or less than about 0.0001 wt %, by weight based on a total weight of the sunscreen composition. In certain embodiments, an emulsifier may be present in the sunscreen composition disclosed herein in an amount ranging from 0 wt % to about 2 wt %, such as from about 0.0001 wt % to about 1.5 wt %, about 0.001 wt % to about 1 wt %, about 0.01 wt % to about 1 wt %, about 0.01 wt % to about 0.5 wt %, about 0.01 wt % to about 0.25 wt %, about 0.01 wt % to about 0.1 wt %, about 0.1 wt % to about 1.5 wt %, about 0.1 wt % to about 1 wt %, about 0.1 wt % to about 0.5 wt %, about 0.1 wt % to about 0.25 wt %, or about 0.0001 wt % to about 0.001 wt %, by weight based on a total weight of the composition.

According to certain embodiments disclosed herein, the water-resistant sunscreen composition may be free of or substantially free of a film former, such as a water resistance film former, and in certain embodiments, the water-resistant sunscreen composition may comprise at least one film former. The film former, when present, may be chosen from any film former known in the art. Film formers may be used to ensure even coverage of certain ingredients, such as UV filters, and can be used to render the sunscreen composition water resistant.

In one aspect, the film former is a hydrophobic material that imparts film forming and/or waterproofing characteristics to the sunscreen composition. For example, one such film former is a polyethylene having a molecular weight of 400. Another suitable film former is a polyethylene with a molecular weight of 2000. Yet another suitable film former is synthetic wax. Other exemplary film formers may include acrylates/acrylamide copolymer, acrylates copolymer, acrylates/C₁₂-C₂₂ alkylmethacrylate copolymer, polyethylene, waxes (e.g., beeswax), VP/dimethiconylacrylate/polycarbamylpolyglycol ester, butylated PVP, PVP/hexadecene copolymer, octadecene/MA copolymer, PVP/eicosene copolymer, tricontanyl PVP, Brassica campestris/aleuritis Fordi Oil copolymer, decamethyl cyclopentasiloxane, trimethylsiloxysilicate, maleated soybean oil glyceryl/octyldodecanol esters, VP/hexadecene copolymers, VP/eicosene copolymers, hydrogenated polyisobutene, cetyl dimethicone, trimethylsiloxysilicate, dimethicone, acrylates/dimethicone copolymers, and mixtures thereof.

In one aspect, the film former included in or excluded from the sunscreen compositions disclosed herein are not soluble in ethanol (such as PVP/Eicosene copolymer). For example, a film former employed in ethanol-based sunscreen products may be Dermacryl® LT or Dermacryl® 79 marketed by Akzo Nobel (INCI Name: acrylates/octylacrylamide copolymer). Dermacryl® LT (CAS Number: 80570-62-3) is a hydrophobic, high molecular weight carboxylated acrylic copolymer, and it may function as a film former in a broad range of cosmetic formulations, imparting waterproofing, increased occlusion and decreased rub-off of actives.

When present, the at least one film former may be present in the sunscreen compositions disclosed herein in any amount sufficient to impart the desired film forming and/or water resistant properties to the composition. In certain embodiments, the at least one film former is present in the sunscreen composition in an amount ranging from about 0.1 wt % to about 10 wt %, such as about 0.5 wt % to about 5 wt %, by weight based on a total weight of the sunscreen composition. In certain embodiments, the sunscreen composition may contain less than about 2 wt % of a film former, such as about 1.5 wt % or less, about 1 wt % or less, less than about 1 wt %, less than about 0.5 wt %, less than about 0.25 wt %, less than about 0.2 wt %, less than about 0.1 wt %, less than about 0.05 wt %, less than about 0.001 wt %, or less than about 0.0001 wt %, by weight based on a total weight of the sunscreen composition. In certain embodiments, at least one film former may be present in the sunscreen composition disclosed herein in an amount ranging from 0 wt % to about 2 wt %, such as from about 0.0001 wt % to about 1.5 wt %, about 0.001 wt % to about 1 wt %, about 0.01 wt % to about 1 wt %, about 0.01 wt % to about 0.5 wt %, about 0.01 wt % to about 0.25 wt %, about 0.01 wt % to about 0.1 wt %, about 0.1 wt % to about 1.5 wt %, about 0.1 wt % to about 1 wt %, about 0.1 wt % to about 0.5 wt %, about 0.1 wt % to about 0.25 wt %, or about 0.0001 wt % to about 0.001 wt %, by weight based on a total weight of the composition.

In certain embodiments disclosed herein, the sunscreen composition comprises at least one UV active ingredient. The at least one UV active ingredient may be selected from any UV active ingredient, and in certain embodiments, the UV active ingredient is a metal oxide. The metal oxide may be selected from oxides of Ba, Zn, Ti, V, Cr, Mn, Fe, Co, Ni, Cu, Re, Os, Jr, and Sn. Non-limiting examples of metal oxides include titanium oxide (amorphous or crystallized in rutile and/or anatase form), iron oxide, zinc oxide, zirconium oxide, and cerium oxide. The metal oxides may comprise treated or untreated metal oxides. Treated metal oxides typically have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen or elastin), alkanolamines, silicon oxides, metal oxides, sodium hexametaphosphate, alumina, or glycerol. The metal oxide may have a mean particle size of about 5 nm to about 30 μm. In some instances, the mean particle size of the metal oxide may be about 5 nm to about 25 μm, about 10 nm to about 10 μm, or about 15 nm to about 5 μm. In further instances, the metal oxide may have a mean particle size of about 5 nm to about 100 nm, about 5 nm to about 75 nm, or about 10 nm to 50 nm. Larger particles sizes may also be useful, such as, e.g., from about 1 μm to about 25 μm, about 5 μm to about 20 μm, or about 10 μm to about 15 μm. The treatment metal oxides may having a coating formed thereon. In certain embodiments, the metal oxide comprises zinc oxide and/or titanium oxide. The zinc oxide may be selected from treated and/or coated zinc oxide. Additionally or alternatively, the titanium oxide may be selected from treated and/or coated titanium oxide. Examples of treated and/or coated zinc oxides include zinc oxides coated with silica and polymethylhydrogenosiloxane, zinc oxides coated with polymethylhydrogenosiloxane, zinc oxides coated with perfluoroalkyl phosphate and copolymer based on perfluoroalkylethyl as a dispersion in cyclopentasiloxane, and zinc oxides coated with silica and polymethylsilsesquioxane. Examples of treated and/or coated titanium oxides include those treated with: silica and alumina; alumina and aluminum stearate; iron oxides and iron stearate; silica, alumina and silicone; sodium hexametaphosphate; octyltrimethoxysilane; alumina and stearic acid; alumina and glycerol; alumina and silicone; and combinations thereof.

In addition to or instead of a metal oxide, the at least one UV active ingredient may be selected from other UV active ingredients known in the art, such as, for example, octinoxate, octisalate, homosalate, avobenzone, octocrylene, para-aminobenzoic acid (PABA), cinoxate, dioxybenzone, menthyl anthranilate, padimate O, ensulizole, sulisobenzone, trolamine salicylate, ecamsule, 4-methylbenzylidene camphor, diethylamino hydroxybenzoyl hexyl benzoate, bis diethylamino hydroxybenzoyl benzoate, disodium phenyl dibenzimidazole tetrasulfonate, Parsol® SLX (INCI Polysilicone-15), butyl methoxydibenzoylmethane, ethylhexyl methoxycinnamate, ethylhexyl salicylate, benzophenone-3, ethylhexyl dimethyl PABA, phenylbenzimidazole sulfonic acid, benzophenone-4, tris-biphenyl triazine, bis-ethylhexyloxyphenol methoxyphenyl triazine, methylene bis-benzotriazolyl tetramethylbutylphenol, ethylhexyl triazone, methoxypropylamino cyclohexenylidene ethoxyethylcyanoacetate, phenylene bis-diphenyltriazine, isoamyl p-methoxycinnamate, benzylidene camphor sulfonic acid, camphor benzalkonium methosulfate, diethylamino hydroxybenzoyl hexyl benzoate, disodium phenyl dibenzimidazole tetrasulfonate, drometrizole trisiloxane, PEG-25 PABA, polyacrylamidomethyl benzylidene camphor, benzophenone-5, benzophenone-8, benzophenone-8, diethylhexyl butamido triazone, and a combination of two or more thereof.

The at least one UV active ingredient (e.g., zinc oxide) may be present in the sunscreen compositions disclosed herein in any amount sufficient to impart the desired UV filtering capacity of the sunscreen. In certain embodiments, the at least one UV active ingredient is present in the sunscreen composition in an amount ranging from about 5 wt % to about 25 wt %, such as from about 5 wt % to about 15 wt %, by weight based on the total weight of the sunscreen composition. For example, the metal oxide may be present in the sunscreen composition in an amount from about 5 wt % to about 25 wt %, about 7 wt % to about 25 wt %, about 9 wt % to about 25 wt %, about 11 wt % to about 25 wt %, about 13 wt % to about 25 wt %, about 15 wt % to about 25 wt %; from about 5 wt % to about 21 wt %, about 7 wt % to about 21 wt %, about 9 wt % to about 21 wt %, about 11 wt % to about 21 wt %, about 13 wt % to about 21 wt %, about 15 wt % to about 21 wt %; from about 5 wt % to about 18 wt %, about 7 wt % to about 18 wt %, about 9 wt % to about 15 wt %, about 11 wt % to about 18 wt %, about 13 wt % to about 18 wt %, about 15 wt % to about 18 wt %; from about 5 wt % to about 15 wt %, about 7 wt % to about 15 wt %, about 9 wt % to about 15 wt %, about 11 wt % to about 15 wt %, about 13 wt % to about 15 wt %; from about 5 wt % to about 13 wt %, about 7 wt % to about 13 wt %, about 9 wt % to about 13 wt %, or about 11 wt % to about 13 wt %, based on the total weight of the sunscreen composition.

In certain embodiments, the water-resistant sunscreen disclosed herein may further comprise at least one photostabilizing agent. Traditional sunscreen compositions may be photo unstable due to the UV active ingredients that are incorporated into such compositions. Accordingly, a photostabilizing agent may be added to the sunscreen composition to stabilize photosensitive ingredients, e.g., UV active ingredients. The photostabilizing, when present, can be selected from octocrylene, diethylhexyl naphthalate, undecylcrylene dimethicone, ethylhexyl methoxycrylene, polyester-8, polyester 25, trimethoxybenzylidene pentanedione, diethylhexyl syringlidene malonate, tetramethyl hydroxy piperidinol, benzotriazole dodecyl p-cresol, sodium benzotriazole butyl phenol sulfonate, butyloctyl salicylate, and combinations thereof. In some cases, the photostabilizer is ethylhexyl methoxycrylene, butyloctyl salicylate, or combinations thereof.

Other exemplary photostabilizers that can be used in the compositions of the disclosure can be selected from Tinosorb® S (USAN bemotrizinol, INCI bis-ethylhexyloxyphenol methoxyphenyl triazine); Tinosorb® M (USAN bisoctrizole, INCI methylene bis-benzotriazolyl tetramethylbutylphenol); hexadecyl benzoate; butyloctyl benzoate; diethylhexyl 2,6-naphthalate; ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, alpha-cyanodiphenylacrylates, and combinations thereof.

The at least one photostabilizer may be present in the sunscreen composition in an amount ranging from about 1 wt % to about 15 wt %, such as from about 1 wt % to about 10 wt %, about 1 wt % to about 8 wt %, about 1 wt % to about 7.5 wt %, about 1 wt % to about 5 wt %, about 1 wt % to about 2.5 wt %, about 2 wt % to about 10 wt %, about 2 wt % to about 8 wt %, about 2 wt % to about 7.5 wt %, about 2 wt % to about 5 wt %, about 2 wt % to about 2.5 wt %; 5 wt % to about 10 wt %, about 5 wt % to about 8 wt %, or about 5 wt % to about 7.5 wt %, based on the total weight of the sunscreen composition.

In certain aspects, the composition disclosed herein can comprise a cosmetically acceptable carrier. The phrase “cosmetically acceptable” means that the material is compatible with skin and hair. For example, “cosmetically acceptable carrier” means a carrier that is compatible with skin and hair and is acceptable for application to the body.

The cosmetically acceptable carrier may include, for example, water and/or water-soluble solvents. Non-limiting examples of cosmetically acceptable carriers include glycerin, C₁₋₄ alcohols, organic solvents, fatty alcohols, fatty ethers, fatty esters, polyols, glycols, vegetable oils, mineral oils, liposomes, laminar lipid materials, water, or any combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

In some instances, cosmetically acceptable carriers may comprise water, a mixture of water and at least one cosmetically acceptable organic solvent, or at least one cosmetically acceptable organic solvent. Additionally, cosmetically acceptable carriers may be or may include ethanol, a glycol ether, for example, dipropylene glycol n-butyl ether, isododecane, mineral oil, propylene glycol, pentylene glycol, hexylene glycol, glycerol, and mixtures thereof.

The total amount of cosmetically acceptable carrier can vary, but in some instances may be about 25 wt % to about 95 wt %, based on the total weight of the sunscreen composition. In some instances, the total amount of cosmetically acceptable carrier is about 35 wt % to about 85 wt %, about 40 wt % to about 85 wt %, about 45 wt % to about 85 wt %, about 25 wt % to about 80 wt %, about 30 wt % to about 80 wt %, about 35 wt % to about 80 wt %, about 40 wt % to about 80 wt %, or about 45 wt % to about 60 wt %, based on the total weight of the sunscreen composition.

In certain aspects the sunscreen composition disclosed herein can comprise one or more oils and/or emollients. For example, oils/emollients that may be included in the sunscreen compositions can be selected from any of the following: hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, coriander oil, castor oil, avocado oil, caprylic/capric acid triglycerides, jojoba oil, shea butter oil and caprylyl glycol; C₁₅-C₁₉ alkanes; synthetic esters and ethers of fatty acids, for instance purcellin oil, octyldodecyl neopentanoate, 2-octyldodecyl stearate, 2-octyldodecyl erucate, isostearyl isostearate; hydroxylated esters, for instance isostearyl lactate, octyl hydroxystearate, octyldodecyl hydroxystearate, glyceryl behenate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C₁₁) and of n-tridecane (C₁₃); fluoro oils that are partially hydrocarbon-based and/or silicone-based; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, polymethylphenylsiloxanes; PEG-20 methyl glucose sesquistearate, and mixtures thereof.

In further aspects, additional specific examples of oils/emollients in the sunscreen compositions of the disclosure may be selected from coco-caprylate/caprate, cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, isopropyl lauroyl sarcosinate, and any combinations thereof.

In further aspects, any of the sunscreen compositions disclosed herein can include oleaginous esters, ethers, and aloe extracts, and mixtures thereof. For example, emollients that can be included in the sunscreen compositions include: Cetiol® OE, Lexol® IPL, octyl palmitate, isopropyl palmitate, neopentyl glycol heptanoate, neopentyl glycol diheptanoate, actiphyte of aloe vera, Trivent® NP-13, and C₁₂₋₁₅ alkyl benzoate.

In some aspects, the sunscreen compositions disclosed herein can include one or more conventional cosmetic oils, such as a mineral oil; hydrogenated polyisobutene; synthetic or natural-made squalane; cosmetic esters or ethers, which may be branched or unbranched, saturated or unsaturated; vegetable oils; or mixtures of two or more thereof.

Particularly suitable oils are vegetable oils with high proportions of omega-3 and omega-6 fatty acids, for example nut oils such as hazelnut oil. Other vegetable oils may also be used, such as calendula oil, jojoba oil, avocado oil, macadamia nut oil, castor oil, cocoa butter, coconut oil, corn oil, cottonseed oil, olive oil, palm kernel oil, rapeseed oil, safflower oil, sesame oil, soybean oil, sunflower seed oil, wheat germ oil, grapeseed oil, kukui nut oil, thistle oil and mixtures from that.

In certain embodiments of the disclosure, the sunscreen composition comprises emollients selected from at least one of dimethicone, coco-caprylate/caprate, isododecane, C₁₅-C₁₉ alkanes (e.g., Jeechem® NDA), and combinations thereof. In certain embodiments of the disclosure, the sunscreen composition comprises emollients selected from at least one of dimethicone, coco-caprylate/caprate, C₁₅-C₁₉ alkanes (e.g., Jeechem® NDA), and combinations thereof, and the sunscreen composition is free of or substantially free of isododecane.

The total amount of oils and/or emollients in the sunscreen composition, if present, may range from about 1 wt % to about 50 wt %, based on the total weight of the sunscreen composition. In some instances, the total amount of oils and/or emollients may range from about 1 wt % to about 25 wt %, such as from about 2 wt % to about 20 wt %, about 3 wt % to about 20 wt %, about 4 wt % to about 20 wt %, about 6 wt % to about 18 wt %, about 8 wt % to about 16 wt %, or about 10 wt % to about 15 wt %, based on the total weight of the sunscreen composition.

In certain aspects, the sunscreen compositions of the disclosure comprise additional active or inactive ingredients, such as humectants, skin conditioning agents, preservatives, pH adjusters, dispersants (e.g., polyhydroxystearic acid), fragrances, dyes, antioxidants, chelating agents, etc.

The sunscreen composition may, optionally, include at least one skin conditioning agent. The conditioning agent may be a conditioning oil, such as a hydrocarbon oil, polyolefins, a fatty ester, silicones, etc. In certain embodiments, the at least one skin conditioning agent is selected from amino acids such as taurine, arginine, glycine, serine, and lysine; polysilicones (e.g., polysilicone 11); ceramides; and bisabolol. Exemplary skin conditioning agents may include Gransil® PC-12 and Symrelief® 100.

The sunscreen composition may further include at least one humectant. The humectant may be selected from various humectants known in the art, including, for example, ascorbyl dipalmitate, acetamide MEA, glucose glutamate, glucuronic acid, TEA-lactate, TEA-PCA, corn syrup, fructose, glucose, glycerin, glycol, 1,2,6-hexanetriol, sodium lactate, sodium PCA, hydrogenated starch hydrolysate, inositol, lactic acid, lactose, mannitol, PCA, PEG-10 propylene glycol, polyamino sugar condensate, propylene glycol, pyridoxine dilaurate, saccharide hydrolysate, hydroxystearyl methylglucamine, glucamine, maltitol, mannitol, methyl gluceth-10, methyl gluceth-20, riboflavin, PEG-4, PEG-6, PEG-8, PEG-9, PEG-10, PEG-12, PEG-14, PEG-16, PEG-18, PEG-20, PEG-32, PEG-40, glutamic acid, glycereth-7, glycereth-12, glycereth-26, saccharide isomerate, sorbeth-20, sorbitol, sucrose, thioglycerin, tris-(hydroxymethyl) nitromethane, tromethamine, histidine, PEG-75, PEG-135, PEG-150, PEG-200, PEG-5 pentaerythritol ether, polyglyceryl sorbitol, sorbitol, urea, xylitol, or the like, or combinations thereof.

In still a further aspect, any of the sunscreen compositions disclosed herein can include one or more antioxidants. For example, antioxidants that can be used in the compositions of the disclosure can be selected from vitamins such as Vitamin C and derivatives thereof, for example, ascorbyl acetate, phosphate and palmitate, magnesium ascorbyl phosphate; Vitamin A and derivatives thereof; folic acid and its derivatives, vitamin E and its derivatives, such as tocopheryl acetate; flavones or flavonoids; amino acids such as histidine, glycine, tyrosine, tryptophan and derivatives thereof; imidazoles such as cis- or trans-urocanic acid and their derivatives; peptides such as D, L-carnosine, D-carnosine, L-carnosine and their derivatives; carotenoids and carotenes, such as α-carotene, β-carotene, lycopene; uric acid and derivatives thereof; α-hydroxy acids such as citric acid, lactic acid, malic acid; α-hydroxy fatty acids such as palmitic acid, phytic acid, lactoferrin; stilbenes and their derivatives; mannose and its derivatives; lipoic acid and its derivatives, e.g., dihydrolipoic acid; ferulic acid and its derivatives; thiols such as glutathione, cysteine, cystine and their esters; and combinations thereof.

In still another aspect, antioxidants or radical scavengers that can be included in any of the sunscreen compositions disclosed herein are plant extracts. For example, antioxidant plant extracts can be those selected from Angelica archangelica, Arnica montana, Camellia sinensis, Cupressus semper, Coffee arabica, Polygonatum multiflorum, Pongamia pinnata, Rosmarinus officinalis, Evernia fufuracea, Evernia prunastri, Aventa sativa, and a mixture of two or more thereof.

In yet another aspect, any of the sunscreen compositions disclosed herein can comprise one or more vitamins and/or antioxidants selected from vitamin A (e.g., retinyl palmitate) and their derivatives, vitamin B1 (e.g., thiamine) and their derivatives, vitamin B2 (e.g., riboflavin) and their derivatives, vitamin B3 (e.g., niacin or nicotinamide/niacinamide) and their derivatives, vitamin B5 (e.g., pantothenic acid) and their derivatives, vitamin B6 (e.g., pyridoxine, pyridoxal, or pyridoxamine) and their derivatives, vitamin B12 (e.g., cyanocobalamin) and their derivatives, vitamin C (e.g., ascorbic acid) and their derivatives, vitamin E (e.g., tocopherol acetate, alpha tocopherol) and their derivatives, vitamin H (e.g., biotin) and their derivatives, vitamin K (e.g., phytonadione) and their derivatives, and vitamin M (e.g., folic acid) and their derivatives, ubiquinone (also known as coenzyme Q10), glutathione, melatonin, resveratrol, citric acid, gallic acid and its esters, nordihydroguaiaretic acid, lipoic acid, dihydrolipoic acid, glycolic acid, alpha-arbutin, chitosan salicylate, rosmarinic acid, green tea extract, smithsonite extract, Angelica archangelica extract, Camellia sinensis extract, Coffee arabica extract, Pongamia pinnata extract, and mixtures thereof.

In yet a further aspect, any of the sunscreen compositions disclosed herein can comprise thickening agents or gelling agents, including substances that can increase the viscosity of the composition. Thickeners include those that can increase the viscosity of a composition without substantially modifying the efficacy of the active ingredient(s) within the sunscreen composition. Thickeners may also increase the stability of the sunscreen compositions disclosed herein.

Non-limiting examples of additional thickening agents that can be used in the context of the present disclosure include carboxylic acid polymers, crosslinked polyacrylate polymers (e.g., polyacrylate crosspolymer-6; polyacrylate-13), polyacrylamide polymers, polysaccharides, waxes (e.g., beeswax), ceramides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Non-limiting examples of crosslinked polyacrylate polymers include cationic and nonionic polymers.

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids. In certain embodiments, the thickener may be selected from hydroxyethyl acrylate/acrylodimethyl taurate copolymer, squalene, polysorbate 60, and combinations thereof (e.g., Simulgel® NS).

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. For example, these can include polymers that are ethers of C₁₀-C₃₀ straight or branched chain alcohols with hydroxyalkylcelluloses. In certain embodiments, the thickener may be an esterified dextrin, such as dextrin palmitate.

Non-limiting examples of gums that can be used with the sunscreens of the present disclosure include acacia (e.g., acacia senegal gum), agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

In the sunscreen compositions disclosed herein, the at least one thickening agent may be present in an amount ranging from about 0.1 wt % to about 25 wt %, such as from about 0.1 wt % to about 10 wt %, about 0.1 wt % to about 5 wt %, about 0.1 wt % to about 4 wt %, about 0.1 wt % to about 3 wt %, about 0.1 wt % to about 2 wt %, about 0.5 wt % to about 10 wt %, about 0.5 wt % to about 5 wt %, about 0.5 wt % to about 4 wt %, about 0.5 wt % to about 3 wt %, about 0.5 wt % to about 2 wt %, about 1 wt % to about 10 wt %, about 1 wt % to about 5 wt %, about 1 wt % to about 4 wt %, about 1 wt % to about 3 wt %, about 1 wt % to about 2 wt %, about 1.5 wt % to about 10 wt %, about 1.5 wt % to about 5 wt %, about 1.5 wt % to about 4 wt %, about 1.5 wt % to about 3 wt %, or about 1.5 wt % to about 2 wt %, based on the total weight of the sunscreen composition.

In some aspects, the form of the sunscreen compositions disclosed herein may be a cream, a gel, a paste, a lotion, a rinse, a foam, an emulsion, a spray, etc. The sunscreen compositions may be prepared according to techniques that are well-known to those skilled in the art, in particular those intended for the preparation of emulsions of oil-in-water or water-in-oil type. For example, they may be in the form of a simple or complex emulsion (O/W, W/O, O/W/O or W/O/W emulsion) such as a cream or a milk, in the form of a gel or a cream-gel, or in the form of a lotion. In certain embodiments, the sunscreen composition disclosed herein is water-resistant sunscreen composition in the form of an oil-in-water emulsion.

In another aspect, the disclosure provides a method of making a water-resistant sunscreen having an SPF of at least about 30, the method comprising formulating an oil-in-water emulsion by contacting at least one metal oxide, at least one alkane, and at least one photostabilizer to formulate a water-resistant sunscreen, wherein the water-resistant sunscreen is free or substantially free of an emulsifier. In certain embodiments disclosed herein, the water-resistant sunscreen composition is free or substantially free of a film former.

In another aspect, the disclosure provides the use of a water-resistant sunscreen composition as disclosed herein, for use in preventing or mitigating damage to the skin caused by UV light, e.g., sunburn. The methods disclosed herein may comprise applying the water-resistant sunscreen composition disclosed herein to a surface of the body, such as the skin, and allowing the water-resistant sunscreen composition to dry. In certain embodiments, the water-resistant sunscreen composition disclosed herein provides SPF protection to the user in an amount of at least about 30, such as at least about 35, at least about 40, or at least about 45. In certain embodiments, the sunscreen composition provides SPF protection to the user before, during, and/or after the user is exposed to water, e.g., in a swimming pool, lake, ocean, or the like. In certain embodiments, the sunscreen composition provides SPF protection to the user in or out of water for a time period of at least about 30 minutes, such as at least about 35 minutes, at least about 40 minutes, at least about 45 minutes, at least about 50 minutes, at least about 55 minutes, or at least about 1 hour.

The sunscreen composition disclosed herein is described with reference to illustrative examples, but it is to be understood that the disclosure is not limited to the disclosed embodiments. Alterations and modifications that would occur to one of skill in the art upon reading the specification are also within the scope of the disclosure.

### EXAMPLES

### Example 1—Sunscreen Compositions Containing an Emulsifier

A base formulation (Base 1) was prepared containing an emulsifier, and eight different prototypes of Base 1 were prepared containing various amounts of zinc oxide and various water resistance polymers (i.e., film formers). The eight prototypes are set forth below in Table 1.

TABLE 1
Base 1 prototype sunscreen formulations showing wt % of ingredients

| | | | | | | | | |
|---|---|---|---|---|---|---|---|---|
| Ingredient | #1 | #2 | #3 | #4 | #5 | #6 | #7O | #7P |

Water phase (wt %)

| | | | | | | | | |
|---|---|---|---|---|---|---|---|---|
| Water | 52.38 | 52.48 | 51.98 | 52.5 | 52.3 | 51.23 | 55.5 | 55 |
| Humectants | 4.025 | 4.025 | 4.025 | 3 | 3 | 4.025 | 3.4 | 3.4 |
| pH adjuster | 0.01 | 0.01 | 0.01 | 0.01 | 0.01 | 0.01 | 0.01 | 0.01 |
| Preservative | 1 | 1 | 1 | 1 | 1 | 1 | 1 | 1 |

Oil phase (wt %)

| | | | | | | | | |
|---|---|---|---|---|---|---|---|---|
| Dimethicone | 5.5 | 5.5 | 5.5 | 4 | 3 | 5.5 | 5.5 | 5.5 |
| Trimethylsiloxysilicate | — | — | 1.5 | 1.5 | — | — | — | — |
| VP/eicosene copolymer | 1.5 | 1.5 | 0.5 | — | — | 1.5 | — | — |
| Dimethicone and | — | — | — | 2.5 | 2.5 | — | — | — |
| dimethicone/acrylates | | | | | | | | |
| copolymer | | | | | | | | |
| Trimethylsiloxysilicate | — | — | — | — | 2.5 | — | — | — |
| and dimethicone | | | | | | | | |
| Beeswax | — | — | — | — | — | — | 2 | — |
| Triacontanyl PVP | — | — | — | — | — | — | — | 1.5 |
| Antioxidant | 0.06 | 0.06 | 0.06 | 0.06 | 0.06 | 0.06 | 0.06 | 0.06 |
| Photostabilizers | 7.5 | 7.5 | 7.5 | 7.5 | 7.5 | 7.5 | 7.5 | 7.5 |
| Octyldodecyl | 2 | 2 | 2 | 2 | 2 | 2 | 2 | 2 |
| neopentanoate | | | | | | | | |
| Caprylic/capric | 3 | 3 | 3 | 3 | 3 | 3 | 3 | 3 |
| triglyceride | | | | | | | | |
| Oleth-20 (emulsifier) | 0.5 | 0.5 | 0.5 | 0.5 | 0.7 | 0.5 | 0.5 | 0.5 |
| Oleth-3 phosphate | 0.5 | 0.5 | 0.5 | 0.5 | 0.5 | 0.5 | 0.5 | 0.5 |
| (emulsifier) | | | | | | | | |
| Polyhydroxystearic acid | — | — | — | — | — | — | 0.3 | 0.3 |
| Cetearyl alcohol and coco- | — | — | — | — | — | — | 1.25 | 1.25 |
| glucoside (emulsifier) | | | | | | | | |
| Cetearyl alcohol and | — | — | — | — | — | 1.25 | — | — |
| cetearyl glucoside | | | | | | | | |
| (emulsifier) | | | | | | | | |
| ZnO and | 11.88 | 11.88 | 11.88 | 11.88 | 11.88 | 11.88 | 13.43 | 13.43 |
| triethoxycaprylysilane | | | | | | | | |
| Sepiplus ® S | 2.3 | 2.3 | 2.3 | 2.3 | 2.3 | 2.3 | 2.3 | 2.3 |

Post phase (wt %)

| | | | | | | | | |
|---|---|---|---|---|---|---|---|---|
| Water | 5 | 5 | 5 | 5 | 5 | 5 | — | — |
| pH adjuster | 0.3 | 0.3 | 0.3 | 0.3 | 0.3 | 0.3 | 0.2 | 0.2 |
| Taladvance ® | 0.2 | — | — | — | — | — | 0.2 | 0.2 |
| Skin conditioning agents | 2.1 | 2.1 | 2.1 | 2.1 | 2.1 | 2.1 | 2.1 | 2.1 |
| Antioxidants | 0.25 | 0.25 | 0.25 | 0.25 | 0.25 | 0.25 | 0.25 | 0.25 |

The SPF values of each prototype were determined following the methodology set forth in 21 C.F.R. § 352.76. Briefly, the sunscreen was applied to a site on the skin (the treated site), allowed to dry, and then panelists were immersed in water with moderate activity for a total of 40 minutes. The panelists were then allowed to air dry, and the treated site was irradiated with UV light to determine the SPF.

Stability data and the percent of in vitro water resistance was also determined. The pass or fail evaluation for stability performance was based on the minimum requirements as forth based on International Council for Harmonisation of Technical Requirements of Pharmaceuticals Human Use (ICH) and World Health Organization (WHO) guidelines for stability studies.

The in vitro water resistance percentage was determined according to the following formulation: in vitro water resistance percentage=(initial SPF−final SPF)/initial SPF*100. The initial SPF was determined using a Labsphere UV-2000S UV analyzer. Each formulation was applied to a PMMA plate at a dose of 1.2 mg/cm². The formulations were then spread evenly in a circular motion followed by a horizontal spreading motion onto the PMMA plate. Once the formulations were uniformly spread they were allowed to dry for 30 minutes. After drying the treated plates were analyzed by the Labsphere UV-2000S UV analyzer, and each plate was measured 9 times at separate locations in order to determine the initial SPF. After the initial SPF determination, the plates were submerged into a water bath with a range of 23-32° C. and a target temperature of 30° C. The water was gently mixed with a flat mixing blade at 50 rpm. The treated plates remained submerged for 80 minutes. After 80 minutes the plates were removed from the water and allowed to air dry for 30 minutes. Afterwards, the plates were analyzed again using the Labsphere UV-2000S to determine the final SPF.

The results are set forth below in Table 2.

TABLE 2
SPF, Stability, and Water Resistance data

| Performance | #1 | #2 | #3 | #4 | #5 | #6 | #7O | #7P |
|---|---|---|---|---|---|---|---|---|
| Stability | Fail | Fail | Pass | Pass | Pass | Fail | Fail | Fail |
| In vitro WR % | 15.86 | — | −67.98 | −11.39 | −36.52 | — | −26.77 | −43.95 |
| 40 min. WR | — | <30 | <30 | — | — | — | — | — |
| SPF (in vivo) | | (TF) | (TF) | | | | | |

As shown in Table 2, none of the prototypes from Base 1 containing an emulsifier were able to provide a water-resistant SPF of at least 30.

### Example 2—Sunscreen Compositions without an Emulsifier

A base formulation (Base 2) was prepared containing no emulsifier, and 15 different prototypes of Base 2 were prepared containing little to no emulsifier, various amounts of zinc oxide, and various water resistance polymers (i.e., film formers). The 15 prototypes are set forth below in Tables 3 and 4.

TABLE 3
Base 2 prototype sunscreen formulations #7-#7.0G showing wt % of ingredients

| | | | | | | | | |
|---|---|---|---|---|---|---|---|---|
| Ingredient | #7 | #7.0A | #7.0B | #7.0C | #7.0D | #7.0E | #7.0F | #7.0G |

Water phase (wt %)

| | | | | | | | | |
|---|---|---|---|---|---|---|---|---|
| Water | 52.88 | 52.97 | 52.91 | 52.91 | 52.91 | 52.91 | 52.91 | 52.91 |
| Thickener | 0.5 | 0.5 | 0.5 | 0.5 | 0.5 | 0.5 | 0.5 | 0.5 |
| Humectants | 2.4 | 2.4 | 2.4 | 2.4 | 2.4 | 2.4 | 2.4 | 2.4 |
| Skin conditioning agents | 2 | 2 | 2 | 2 | 2 | 2 | 2 | 2 |
| Preservative | 1 | 1 | 1 | 1 | 1 | 1 | 1 | 1 |
| EDTA | 0.05 | 0.05 | 0.05 | 0.05 | 0.05 | 0.05 | 0.05 | 0.05 |
| pH adjuster | 0.29 | 0.29 | 0.29 | 0.29 | 0.29 | 0.29 | 0.29 | 0.29 |

Oil phase (wt %)

| | | | | | | | | |
|---|---|---|---|---|---|---|---|---|
| Dimethicone | 4.44 | 4.44 | 4 | 4 | 4 | 4 | 4 | 4 |
| Coco-caprylate/caprate | 8 | 8 | 7 | 7 | 7 | 7 | 7 | 7 |
| Butyloctyl salicylate | 5 | 5 | 5 | 5 | 5 | 5 | 5 | 5 |
| Ethylhexyl | 2.5 | 2.5 | 2.5 | 2.5 | 2.5 | 2.5 | 2.5 | 2.5 |
| methoxycrylene | | | | | | | | |
| Maleated soybean oil | — | — | 1.5 | — | — | — | — | — |
| glyceryl/octododecanol | | | | | | | | |
| esters | | | | | | | | |
| Triacontanyl PVP | — | — | — | 1.5 | — | 0.75 | — | — |
| VP/hexadecene copolymer | — | — | — | — | 1.5 | 0.75 | — | — |
| VP/eicosene copolymer | — | — | — | — | — | — | 1.5 | — |
| Hydrogenated | — | — | — | — | — | — | — | 1.5 |
| polyisobutene | | | | | | | | |
| Antioxidants | 0.31 | 0.31 | 0.31 | 0.31 | 0.31 | 0.31 | 0.31 | 0.31 |
| Dispersant | 0.8 | 0.8 | 0.8 | 0.8 | 0.8 | 0.8 | 0.8 | 0.8 |
| Isododecane | 3 | — | — | — | — | — | — | — |
| Jeechem ® NDA (C15-C19 | — | 3 | 3 | 3 | 3 | 3 | 3 | 3 |
| alkanes) | | | | | | | | |
| ZnO and | 13.43 | 13.43 | 13.43 | 13.43 | 13.43 | 13.43 | 13.43 | 13.43 |
| triethoxycaprylysilane | | | | | | | | |
| Thickeners | 0.70 | 0.70 | 0.70 | 0.70 | 0.70 | 0.70 | 0.70 | 0.70 |
| Skin conditioning agents | 2.1 | 2.1 | 2.1 | 2.1 | 2.1 | 2.1 | 2.1 | 2.1 |

Post phase (wt %)

| | | | | | | | | |
|---|---|---|---|---|---|---|---|---|
| Thickeners | 0.6 | 0.6 | 0.6 | 0.6 | 0.6 | 0.6 | 0.6 | 0.6 |
| Total emollient: | 15.44 | 15.44 | 14 | 14 | 14 | 14 | 14 | 14 |
| Total photostabilizer: | 7.5 | 7.5 | 7.5 | 7.5 | 7.5 | 7.5 | 7.5 | 7.5 |
| Total thickener: | 1.2 | 1.2 | 1.2 | 1.2 | 1.2 | 1.2 | 1.2 | 1.2 |
| Total film formers: | 0 | 0 | 1.5 | 1.5 | 1.5 | 1.5 | 1.5 | 1.5 |
| Total emulsifier: | 0 | 0 | 0 | 0 | 0 | 0 | 0 | 0 |

TABLE 4
Base 2 prototype sunscreen formulations #7.0H-#7.0N
showing wt % of ingredients

| | | | | | | | |
|---|---|---|---|---|---|---|---|
| Ingredient | #7.0H | #7.0I | #7.0J | #7.0K | #7.0L | #7.0M | #7.0N |

Water phase (wt %)

| | | | | | | | |
|---|---|---|---|---|---|---|---|
| Water | 52.91 | 52.41 | 51.91 | 52.99 | 53.94 | 53.94 | 52.84 |
| Thickener | 0.5 | 0.5 | 0.5 | 0.5 | 0.5 | 0.5 | 0.5 |
| Humectants | 2.4 | 2.4 | 2.4 | 2.4 | 2.4 | 2.4 | 2.4 |
| Skin conditioning agents | 2 | 2 | 2 | 2 | 2 | 2 | 2 |
| Preservative | 1 | 1 | 1 | 1 | 1 | 1 | 1 |
| EDTA | 0.05 | 0.05 | 0.05 | 0.05 | 0.05 | 0.05 | 0.05 |
| pH adjuster | 0.2 | 0.2 | 0.2 | 0.18 | 0.29 | 0.29 | 0.29 |

Oil phase (wt %)

| | | | | | | | |
|---|---|---|---|---|---|---|---|
| Dimethicone | 4 | 4 | 4 | 5 | 5 | 5 | 5 |
| Coco-caprylate/caprate | 7 | 7 | 7 | 4.88 | 4.88 | 4.88 | 4.88 |
| Butyloctyl salicylate | 5 | 5 | 5 | 5 | 5 | 5 | 5 |
| Ethylhexyl | 2.5 | 2.5 | 2.5 | 3 | 3 | 3 | 3 |
| methoxycrylene | | | | | | | |
| Triacontanyl PVP | — | 1 | 1 | — | — | — | — |
| Cetyl dimethicone | 1.5 | — | — | — | — | — | — |
| Dextrin palmitate | — | — | — | — | 1 | — | — |
| Beeswax | — | — | — | — | — | 1 | 2 |
| Fluidfeel Easy ® | — | — | — | — | — | — | 0.25 |
| Montanov ® 82 | — | 1 | 1.5 | — | — | — | — |
| Antioxidants | 0.31 | 0.31 | 0.31 | 0.31 | 0.31 | 0.31 | 0.31 |
| Polyhydroxystearic acid | 0.8 | 0.8 | 0.8 | 0.8 | 0.8 | 0.8 | 0.8 |
| Jeechem ® NDA (C15-C19 | 3 | 3 | 3 | 3 | 3 | 3 | 3 |
| alkanes) | | | | | | | |
| ZnO and | 13.43 | 13.43 | 13.43 | 13.43 | 13.43 | 13.43 | 13.43 |
| triethoxycaprylysilane | | | | | | | |
| Thickeners | 0.7 | 0.7 | 0.7 | 0.7 | 0.7 | 0.7 | 0.7 |
| Skin conditioning agents | 2.1 | 2.1 | 2.1 | 2.1 | 2.1 | 2.1 | 2.1 |

Post phase (wt %)

| | | | | | | | |
|---|---|---|---|---|---|---|---|
| Thickeners | 0.6 | 0.6 | 0.6 | 0.6 | 0.6 | 0.6 | 0.6 |
| Total emollient: | 14 | 14 | 14 | 12.88 | 12.88 | 12.88 | 12.88 |
| Total photostabilizer: | 7.5 | 7.5 | 7.5 | 8 | 8 | 8 | 8 |
| Total thickener: | 1.2 | 1.2 | 1.2 | 1.2 | 2.2 | 2.2 | 3.05 |
| Total film formers: | 1.5 | 1 | 1 | 0 | 0 | 0 | 0 |
| Total emulsifier: | 0 | 1 | 1.5 | 0 | 0 | 0 | 0.25 |

The SPF values, stability data, and in vitro water resistance of each prototype were determined following the methodologies set forth above in Example 1. The results are set forth below in Table 5.

TABLE 5
SPF, Stability, and Water Resistance data

| Performance | #7 | #7.0A | #7.0B | #7.0C | #7.0D | #7.0E | #7.0F | #7.0G |
|---|---|---|---|---|---|---|---|---|
| Stability | Pass | Pass | Fail | Fail | Fail | Fail | Fail | Fail |
| In vitro WR % | 1.98 | −16.48 | −36.09 | −44.50 | — | −0.23 | — | −12.26 |
| 40 min. WR | 33 | 37 | — | — | — | — | — | — |
| SPF (in vivo) | | | | | | | | |

| | #7.0H | #7.0I | #7.0J | #7.0K | #7.0L | #7.0M | #7.0N |
|---|---|---|---|---|---|---|---|
| Stability | Pass | Fail | Fail | Pass | Fail | Fail | Fail |
| In vitro WR % | −12.96 | — | — | — | 7.14 | 21.91 | −4.48 |
| 40 min. WR | — | — | — | 43 | — | — | 45 |
| SPF (in vivo) | | | | | | | |

As shown in Table 5, four of the prototypes were able to provide a water-resistant SPF of at least 30 (#7, #7.0A, #7.0K, and #7.0N), and three of the prototypes passed the stability testing and had a water-resistant SPF of at least 30 (#7, #7.0A, and #7.0K).

### Example 3—Base 3 Oil-In-Water Prototype Sunscreen Formulation

A third base (Base 3) formulation was prepared as an alternative oil-in-water emulsion that was emulsifier free and contained a different polymer stabilizing system (Sepiplus® 400) from the prototypes prepared in Example 2. The prototype formulation is set forth below in Table 6.

TABLE 6
Base 3 prototype sunscreen formulation 8.0

| | |
|---|---|
| Ingredient | #8.0 |

Water phase (wt %)

| | |
|---|---|
| Water | 53.77 |
| Humectants | 2.4 |
| Skin conditioning agents | 2 |
| Preservative | 1 |

Oil phase (wt %)

| | |
|---|---|
| Isopropyl palmitate | 7.4 |
| Ethylhexyl stearate | 6 |
| Octyldodecyl neopentanoate | 5.7 |
| Hydrogenated polyisobutene | 0.5 |
| Oleth-3 phosphate | 0.29 |
| Antioxidants | 0.25 |
| Skin conditioning agent | 0.1 |
| ZnO/triethoxycaprylysilane | 9.36 |
| TiO₂/aluminum/hydrogen dimethicone | 9.13 |
| Thickener | 2 |

Post phase (wt %)

| | |
|---|---|
| Skin conditioning agent | 0.1 |

The SPF values, stability data, and in vitro water resistance of prototype #8.0 were determined following the methodologies set forth above in Example 1. The results are set forth below in Table 7.

TABLE 7
SPF, Stability, and Water Resistance data

| Performance | #8.0 |
|---|---|
| Stability | Fail |
| In vitro WR % | −58.36 |
| 40 min. WR SPF (in vivo) | — |

As shown in Table 7, prototype #8.0 did not provide adequate stability or water resistance.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

What is claimed is:

1. A water-resistant sunscreen composition comprising an oil-in-water emulsion, wherein the oil-in-water emulsion comprises at least one UV active ingredient, at least one alkane, and at least one photostabilizer and is substantially free of an emulsifier, and
wherein the sunscreen composition provides a water-resistant sun protection factor (SPF) of at least about 30.

2. The water-resistant sunscreen composition according to claim 1, wherein the at least one UV active ingredient is at least one metal oxide.

3. The water-resistant sunscreen composition according to claim 2, wherein the at least one metal oxide is selected from zinc oxide and titanium dioxide.

4. The water-resistant sunscreen composition according to claim 1, wherein the at least one metal oxide is present in an amount ranging from about 10 wt % to about 20 wt %, based on a total weight of the water-resistant sunscreen composition.

5. The water-resistant sunscreen composition according to claim 1, wherein the water-resistant sunscreen composition is substantially free of a film former.

6. The water-resistant sunscreen composition according to claim 1, wherein the at least one photostabilizer is selected from ethylhexyl methoxycrylene, butyloctyl salicylate, or a combination thereof.

7. The water-resistant sunscreen composition according to claim 1, wherein the at least one photostabilizer is present in the water-resistant sunscreen composition in an amount ranging from about 1 wt % to about 15 wt %, based on a total weight of the water-resistant sunscreen composition.

8. The water-resistant sunscreen composition according to claim 1, wherein the at least one photostabilizer comprises ethylhexyl methoxycrylene.

9. The water-resistant sunscreen composition according to claim 1, wherein the at least one alkane is selected from C15-C19 alkanes.

10. The water-resistant sunscreen composition according to claim 1, wherein the water-resistant sunscreen composition is substantially free of isododecane.

11. The water-resistant sunscreen composition according to claim 1, further comprising at least one skin conditioning agent.

12. The water-resistant sunscreen composition according to claim 11, wherein the at least one skin conditioning agent is selected from amino acids, polysilicones, bisabolol, and combinations thereof.

13. The water-resistant sunscreen composition according to claim 1, further comprising at least one antioxidant.

14. The water-resistant sunscreen composition according to claim 13, wherein the at least one antioxidant is selected from vitamin E and derivatives thereof.

15. The water-resistant sunscreen composition according to claim 1, further comprising a cosmetically acceptable carrier.

16. The water-resistant sunscreen composition according to claim 15, wherein the cosmetically acceptable carrier is water, and wherein the water is present in an amount ranging from about 40 wt % to about 60 wt %, based on a total weight of the water-resistant sunscreen composition.

17. A method of preventing or mitigating damage to skin comprising applying the water-resistant sunscreen composition according to claim 1 to the skin of a user in need thereof and allowing the water-resistant sunscreen composition to dry on the skin before exposing the skin to UV radiation.

18. The method according to claim 17, wherein the method provides a water-resistant SPF protection to the user in an amount of at least about 30.

19. The method according to claim 17, wherein the skin of the user is exposed to water.

20. The method according to claim 19, wherein the skin of the user is exposed to water for at least about 40 minutes.
